(19) United States
(12) Patent Application Publication
(10) Pub. No.: US 2023/0329376 A1
(43) Pub. Date: Oct. 19, 2023

(54) MEDICAL HAND GUARD

(71) Applicant: DAVID CHERNOBYLSKY, WOODLAND HILLS, CA (US)

(72) Inventor: DAVID CHERNOBYLSKY, WOODLAND HILLS, CA (US)

(21) Appl. No.: 18/022,075

(22) PCT Filed: Aug. 31, 2021
(86) PCT No.: PCT/US2021/048381

§ 371 Date: Feb. 17, 2023

Related U.S. Application Data
(60) Provisional application No. 63/073,157, filed on Sep. 1, 2020.

(51) Int. Cl. A41D 19/015

(52) U.S. Cl. CPC A41D 19/01505

(57) ABSTRACT

A sterile and disposable medical hand guard includes a dorsal absorption layer and an optional wrist layer. The sharps guard is wearable underneath or above a standard medical glove and includes an adhesive layer for attachment to either the skin of a user's hand or a surface of a medical glove. The dorsal absorption layer is shaped to protect a dorsal thenar space of the hand, and optionally at least a portion of the dorsal regions of each of the index finger and thumb. The dorsal absorption layer includes a semi-fluid gel surrounded by a firm gel to slow the ingress of sharps (e.g., a needle) and provide sufficient time to stop ingress of the sharps prior to reaching the skin of the user's hand. The wrist layer includes gel segments to provide a comfortable pressure around a user's wrist while facilitating wrist flexion and dexterity of motion.

The present application claims the benefit of U.S. Provisional Application No. 63/073,157 filed Sep. 1, 2020 and entitled “MEDICAL HAND GUARD.” the disclosure of which is hereby incorporated by reference in its entirety.

### TECHNICAL FIELD

The present disclosure relates to medical equipment generally and more specifically to protective equipment worn during medical (e.g., surgical) procedures.

### BACKGROUND

During medical (e.g., surgical) procedures, sterile disposable gloves are worn as a barrier to pathogens. However, most modern medical gloves are not needlestick or abrasion proof. Needlestick or abrasion resistant gloves are generally constructed of very thick or technologically complex materials, rendering them non-disposable and non-cost-effective, especially in a sterile environment such as Surgical Operation Rooms or Intensive Care Units. Further, such existing resistant gloves are not flexible enough due to their overall thickness for the proper dexterity and precise handling required during surgical procedures. Even doubleglove techniques have been found to be ineffective against needle-stick punctures.

Punctures and cuts that occur in surgical operation rooms promote the spread of HIV, Hepatitis B, and other blood-borne pathogens that directly affect medical personnel. Sterile disposable gloves are used in modern sterile field surgeries prevent contamination from the hands of the medical staff to a surgical incision. However, there are limited successful solutions available in the opposite direction, for preventing contamination from wounded patients with blood-borne pathogens transmitting the pathogens to surgeons or medical staff.

As such, the available technology is not sufficient to prevent the considerable hospital workplace accidents associated with blood-borne pathogen contamination of medical staff. Specifically, three types of glove barrier failure are known to affect the medical community at large. Micro-tears, or micro-perforations, due to manufacturing malfunctions allow for small viruses and bacteria to cross the barrier and infect medical staff, who would unknowingly potentially pass it onto other hospital patients. Second, small punctures that go unnoticed could also offer another route for blood-borne pathogens to cross the barrier and cause potentially the same effect. The third barrier failure is when a large and visible puncture happens with a contaminated vessel, such as a needle or scalpel containing infected blood.

In addition to the transmission of blood-borne pathogens from patients to medical personnel, surgeons and other medical personnel also run the risk of experiencing arthritis-like symptoms, including pain and aches, due to the long operating hours. These symptoms can jeopardize surgical procedures and patient safety. Further, the cold temperatures of many medical environments, such as operation rooms often maintained at temperatures between 66 and 68 degrees Fahrenheit. can worsen rheumatic pain and aches. Such pain and aching can be exacerbated greatly during long surgical operations, such as those that run around 10-20 hours or more.

### SUMMARY

The term embodiment and like terms are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, supplemented by this summary. This summary is a high-level overview of various aspects of the disclosure and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this disclosure, any or all drawings and each claim.

Embodiments of the present disclosure include a medical hand guard, comprising: a body section shaped to cover at least a portion of an index finger of a hand of a user and at least a portion of a wrist of the user, the body section having a top surface and a bottom surface; a dorsal absorption layer coupled to the top surface of the body section, wherein the dorsal absorption layer comprises gel, and wherein the dorsal absorption layer covers at least a dorsal thenar space of the hand adjacent a thumb and the index finger of the hand; and an adhesive layer coupled to the bottom surface of the body section.

In some cases, the gel of the dorsal absorption layer includes a semi-fluid gel material surrounded by a firm gel material. In some cases, the gel of the dorsal absorption layer has a thickness between 2.5 mils and 4.5 mils. In some cases, the dorsal absorption layer further covers at least a portion of a dorsal region of the index finger and at least a portion of a dorsal region of the thumb. In some cases, the medical hand guard further includes a wrist layer, the wrist layer comprising additional gel, wherein the wrist layer covers at least a portion of the wrist of the user. In some cases, the additional gel of the wrist layer includes an additional firm gel material. In some cases, the wrist layer covers a circumference of the wrist of the user. In some cases, the additional gel comprises a set of parallel slices to facilitate wrist flexion. In some cases, the body section comprises an elastomeric material having a thickness between 0.5 mil to 2.5 mil. In some cases, the medical hand guard further includes a surgical glove covering the body section and dorsal absorption layer, wherein the surgical glove compresses the gel of the dorsal absorption layer against the body section. In some cases, the medical hand guard further includes a surgical glove for covering all digits of the hand, wherein the surgical glove is coupled to the body section via the adhesive layer. In some cases, the medical hand guard is sterile and disposable. In some cases, the dorsal absorption layer comprises a plurality of sublayers, the gel defines at least one of the plurality of sublayers, and a first sublayer of the plurality of sublayers has a first visual characteristic that is distinct from a second visual characteristic of a second sublayer of the plurality of sublayers. In some cases, the first visual characteristic is a first color and the second visual characteristic is a second color that is visually distinguishable from the first color.

Embodiments of the present disclosure include a medical hand guard, comprising: an absorption layer having a top surface and a bottom surface, wherein the absorption layer comprises gel, and wherein the absorption layer is shaped to cover at least a dorsal thenar space of a hand adjacent a thumb and an index finger of the hand; and an adhesive layer coupled to the bottom surface of the absorption layer.

In some cases, the gel of the absorption layer includes a semi-fluid gel material surrounded by a firm gel material. In some cases, the gel of the absorption layer has a thickness between 2.5 mils and 4.5 mils. In some cases, the absorption layer is further shaped to cover at least a portion of a dorsal region of the index finger and at least a portion of a dorsal region of the thumb. In some cases, the medical hand guard further includes comprising a surgical glove for covering all digits of the hand, wherein the surgical glove is coupled to the absorption layer via the adhesive layer. In some cases, the medical hand guard is sterile and disposable. In some cases, the medical hand guard further includes a wrist layer, the wrist layer comprising additional gel, wherein the wrist layer covers at least a portion of the wrist of the user. In some cases, the additional gel of the wrist layer includes an additional firm gel material. In some cases, the wrist layer covers a circumference of the wrist of the user. In some cases, the additional gel comprises a set of parallel slices to facilitate wrist flexion. In some cases, the medical hand guard further includes a surgical glove covering the absorption layer, wherein the surgical glove compresses the gel of the dorsal absorption layer against the hand when worn. In some cases, the dorsal absorption layer comprises a plurality of sublayers, the gel defines at least one of the plurality of sublayers, and a first sublayer of the plurality of sublayers has a first visual characteristic that is distinct from a second visual characteristic of a second sublayer of the plurality of sublayers. In some cases, the first visual characteristic is a first color and the second visual characteristic is a second color that is visually distinguishable from the first color.

Embodiments of the present disclosure A medical hand guard, comprising: a body section shaped to cover at least a portion of an index finger of a hand of a user and at least a portion of a wrist of the user, the body section having a top surface and a bottom surface; a dorsal absorption layer coupled to the top surface of the body section, wherein the dorsal absorption layer comprises a plurality of sublayers, wherein a first sublayer of the plurality of sublayers has a first visual characteristic that is distinct from a second visual characteristic of a second sublayer of the plurality of sublayers, and wherein the dorsal absorption layer covers at least a dorsal thenar space of the hand adjacent a thumb and the index finger of the hand.

In some cases, the first visual characteristic is a first color and the second visual characteristic is a second color that is visually distinguishable from the first color. In some cases, the medical hand guard further comprises an adhesive layer coupled to the bottom surface of the body section. In some cases, the plurality of sublayers includes at least one gel sublayer. In some cases, the at least one gel sublayer includes a semi-fluid gel material surrounded by a firm gel material. In some cases, the dorsal absorption layer further covers at least a portion of a dorsal region of the index finger and at least a portion of a dorsal region of the thumb. In some cases, the medical hand guard further comprises a wrist layer, the wrist layer comprising a plurality of wrist sublayers, wherein a first wrist sublayer of the plurality of wrist sublayers has a first wrist sublayer visual characteristic that is distinct from a second wrist sublayer visual characteristic of a second wrist sublayer of the plurality of wrist sublayers. In some cases, the plurality of wrist sublayers include a set of parallel slices to facilitate wrist flexion.

### BRIEF DESCRIPTION OF THE DRAWINGS

The specification makes reference to the following appended figures, in which use of like reference numerals in different figures is intended to illustrate like or analogous components.

FIG. 1 is an axonometric projection of a medical sharps guard with a dorsal absorption layer, according to certain aspects of the present disclosure.

FIG. 2 is a side view of a medical hand guard with a dorsal absorption layer, according to certain aspects of the present disclosure.

FIG. 3 is a bottom view of a medical hand guard with a dorsal absorption layer, according to certain aspects of the present disclosure.

FIG. 4 is an axonometric projection of a medical hand guard with a dorsal absorption layer and a wrist layer, according to certain aspects of the present disclosure.

FIG. 5 is a bottom view of a medical hand guard including a dorsal absorption layer and a wrist layer coupled to a medical glove, according to certain aspects of the present disclosure.

FIG. 6 is a cross-sectional view of a portion of a medical hand guard and dorsal absorption layer, according to certain aspects of the present disclosure.

FIG. 7 is a cross-sectional view of a portion of a medical hand guard and wrist layer, according to certain aspects of the present disclosure.

FIG. 8 is a flowchart depicting a process for using a medical hand guard, according to certain aspects of the present disclosure.

FIG. 9 is a schematic cross-sectional view of a set of a needles puncturing a dorsal absorption layer, according to certain aspects of the present disclosure.

FIG. 10 is a schematic top view of a set of a needles puncturing a dorsal absorption layer, according to certain aspects of the present disclosure.

### DETAILED DESCRIPTION

Certain aspects and features of the present disclosure relate to a medical hand guard for protecting a hand during surgical or other medical procedures. The sharps guard is designed to be a sterile and disposable product. The sharps guard can be worn underneath or above a standard medical glove (e.g., nitrile surgical glove) and includes an adhesive layer for attachment to either the skin of a user's hand or a surface of a medical glove. The sharps guard includes a dorsal absorption layer and optionally a wrist layer. The dorsal absorption layer is shaped to protect a dorsal thenar space of the hand, and optionally at least a portion of the dorsal regions of each of the index finger and thumb. In some cases, the dorsal absorption layer includes a thickened region of one or more glove layers of sufficient density and thickness to slow the ingress of sharps (e.g., a needle) and provide sufficient time to stop ingress of the sharps prior to reaching the skin of the user's hand. In some cases, the dorsal absorption layer includes a semi-fluid gel surrounded by a firm gel of sufficient density and thickness to slow the ingress of sharps (e.g., a needle) and provide sufficient time to stop ingress of the sharps prior to reaching the skin of the user's hand. In some cases, the wrist layer includes a thickened region of one or more glove layers formed as parallel slices designed to provide pressure around a user's wrist while facilitating wrist flexion and dexterity of motion. In some cases, the wrist layer includes a firm gel comprising parallel slices designed to provide pressure around a user's wrist while facilitating wrist flexion and dexterity of motion.

As used herein, certain aspects of the present disclosure relate to protecting a user (e.g., medical personnel, such as a surgeon) against harm from an accidental puncture, laceration, or other wound associated with a medical sharp, such as a needle or scalpel. As used herein, the term medical sharp is inclusive of any objects in a medical environment that may pose a puncture, laceration, or other wounding hazard to a user. Examples of common medical sharps include needles (e.g., hypodermic needles, suture needles, and the like), lancets, scalpels, chisels, scissors, pointed forceps (e.g., Adson tissue forceps and tenaculum), and other such equipment. Medical sharps can also include broken items found in medical environments, such as broken ampules, broken syringes, and the like, which items may exhibit sharp edges capable of puncture, laceration, or the like.

It has been determined that most punctures and abrasions that occur during medical procedures occur around a specific region of the back of the hand, known in medical communities as the anatomical thenar space. This dorsal thenar space is a region on the back of the hand adjacent the index finger and thumb. Around this region on the dorsum of the hand, as well as along the dorsal side of the index finger and thumb, most punctures happen and risk the transmission of blood-borne pathogens from patients to surgeons or other medical personnel.

Certain aspects and features of the present disclosure include an absorption layer positionable over these regions (e.g., the dorsal thenar space, a dorsal region of the index finger, a dorsal region of the thumb, or any combination thereof). In some cases, the absorption layer can be a thickened region that includes one or more layers of material, such as the same material of which the glove is made or a similar material. In some cases, the absorption layer can include multiple layers each having a different resistance to puncture, such as due to differing thicknesses and/or differing materials. In some cases, the absorption layer can include a gel. As used herein, as appropriate, reference to one or more layers made of a gel can be replaced with one or more layers made of a different material, such as the material of which the glove body is made.

In some cases, the absorption layer can include a gel. The gel can be a singlelayer gel or a multi-layer gel. In some cases, the gel can include a semi-fluid gel surrounded by a firm gel. The gel can be approximately 3-4 mils thick, such as at or around 2.5 mils to 4.5 mils, 3 mils to 4 mils, or 3.25 mils to 3.75 mils, although other thicknesses can be used. The thickness of the gel can be designed to account for the common one-third depth of most 8 mm-length needles (approximately 2.67 mils) insertion per full second in most accident cases. Thus, use of the absorption layer can permit a needle or other medical sharp to engage the absorption layer before engaging the skin of the user. The depth of the absorption layer can slow ingress of the needle or other medical sharp and give the user time to slow, stop, and/or reverse travel of the needle or other medical sharp (e.g., scalpel). The use of an absorption layer can thus increase the time and distance between initial contact of the needle or other medical sharp with the protective equipment (e.g., the absorption layer) and full infiltration of the needle or other medical sharp to and/or through the skin of the user.

In some cases, the absorption layer can be coupled to a body section. The body section can be of an elastomeric material similar to standard medical gloves (e.g., latex, nitrile, and the like) and of a thickness similar to standard medical gloves (e.g., at or around 1-2 mils). The body section can cover the entire hand (e.g., all digits of the hand) or can be designed to cover only a portion of the hand. In some cases, the body section is shaped to cover at least a portion of the thumb (e.g., a dorsal portion), at least a portion of the index finger (e.g., a dorsal portion), a portion of the dorsum of the hand (e.g., a dorsal thenar region), and optionally at least a portion of a wrist (e.g., a dorsal portion of a wrist or a full circumference of a wrist). The body section can be secured in place through use of an adhesive layer. The adhesive layer can attach the body section to the skin of a user or to a surface of a glove worn by the user.

In some cases, the absorption layer is coupled to or otherwise formed into the body section. In some cases, however, the absorption layer is itself coupled to an adhesive layer, allowing the absorption layer to be adhered to the skin of a user's hand, to a glove worn by a user, or to the body section described herein.

In some cases, the gel of the absorption layer can include a firm gel and a semifluid gel. The firm gel can be more viscous than the semi-fluid gel. The semi-fluid gel can comprise a mixture of water-insoluble surfactant, a water-repellant agent, a water-insoluble base, and a binder to bind the mix into a semi-fluid consistency. In some cases, the semi-fluid gel is surrounded by firm gel. The semi-fluid inner gel can provide additional support and an even more durable barrier for a needle or other sharps to pass through to reach a user's skin.

In some cases, the absorption layer can also resist puncture, cutting, or abrasion. The absorption layer can resist puncture, cutting, or abrasion inherently or with use of an additional puncture resistant layer.

Certain aspects of the present disclosure lower puncture accidents in the surgical operation rooms without hindering the dexterity and integrity of the barrier system (e.g., a standard medical glove). Certain aspects of the present disclosure include differential density and thickness at different locations of the hand, allowing for dexterity while also preventing needles and sharp objects from getting through the barrier system.

In some cases, the medical hand guard can include a wrist layer. The wrist layer can provide a support on and/or around the wrist of a user, which can help alleviate pain and aching. The wrist layer can apply moderate pressure to the user's wrist, thus providing support for the wrist and alleviating wrist pains and aches. Due to the sterile and disposable nature of the medical hand guard with a wrist layer, it can be easily used in surgical procedures where a sterile working environment is important, thus providing beneficial pain-alleviating support to working surgeons. Thus, the medical hand guard with a wrist layer can potentially result in better surgical results and better prognoses for patients given that the surgeons would be able to perform operations better without pain and aching wrists.

The wrist layer can include gel, which can be similar to the firm gel of the absorption layer. In some cases, the wrist layer can be contiguous with the absorption layer, although in other cases it is a separate layer. The wrist layer can include a semi-fluid gel in some cases, although that need not always be the case. The gel of the wrist layer can be segmented to improve dexterity of the user. The segmentation of the wrist layer can include separate segments of gel and/or a single piece of gel that has been partially or fully cut through. The cuts and/or separations between segments can be referred to as parallel slices. The parallel slices can be angled with respect to an axial direction of the arm (e.g., a direction extending down the arm, such as a direction approximately parallel the ulna or radius of the arm). The slices can be of any suitable size and frequency. Further, the use of parallel slices can allow for adjustability of pressure on the wrist depending on how high or how low the wrist layer sits on the wrist or forearm.

Certain aspects of the present disclosure can be work either over or under medical gloves, or can be incorporated into a medical glove design. Furthermore, the design of the medical hand guard can facilitate easy donning and removal, especially when attempting to maintain sterile conditions. In some cases, the use of an adhesive layer can permit the disclosed medical hand guard to adhere to skin (e.g., skin that has been recently scrubbed and disinfection in preparation for surgery) and/or a surgical glove (e.g., a nitrile glove). In some cases, a medical hand guard as disclosed herein can be worn under an outer glove (e.g., a medical glove), in which case the outer glove can compress the gel of the absorption layer and/or the wrist layer, which can improve comfort for some users.

Certain aspects of the present disclosure facilitate alleviation, rather than constriction, of rheumatic wrist pressure. Certain aspects of the present disclosure increase protection to regions of the hand that are at high risk of glove barrier failure (e.g., due to inadvertent contact with medical sharps) while leaving other regions thin enough to maintain high dexterity. In some cases, certain aspects of the present disclosure can be used on either a left or right hand.

In some cases, the absorption layer can include thin regions and thick regions, with the thin regions located to prevent loss of dexterity and the thick regions located to enhance protection in regions at high risk of glove barrier failure.

In some cases, the medical hand guard can include one or more visual indicators to help align the medical hand guard on the hand. For example, a dorsal absorption layer can include one or more visual indicators to aid in aligning the dorsal absorption layer on the hand and over the dorsal thenar space.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

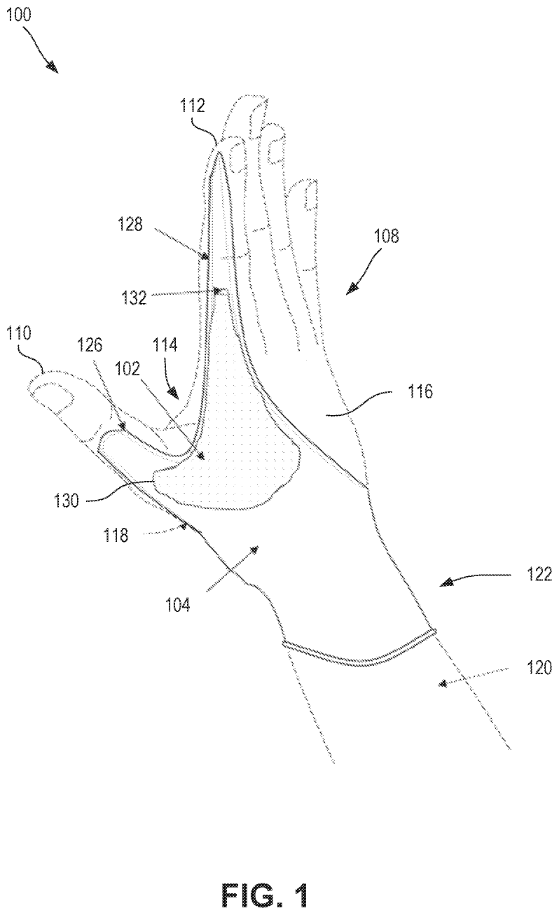

FIG. 1 is an axonometric projection of a medical hand guard 100 with a dorsal absorption layer 102, according to certain aspects of the present disclosure. The medical hand guard 100 is designed to be worn on a hand 108 of a user. The hand 108 can include a thumb 110 and index finger 112. The hand 108 can be coupled to an arm 120 by a wrist 122. The medical hand guard 100 is depicted as adhered to the hand 108 using an adhesive layer (not shown). In other cases, however, the medical hand guard 100 can be adhered to a standard medical glove or incorporated into a medical glove.

The medical hand guard 100 can include a body section 104. The body section 104 can be made of a elastomeric material, such as a material similar to that of a standard medical glove (e.g., latex or nitrile). The body section 104 can have a thickness at or around 1 to 2 mils, although other thicknesses can be used. In some cases, the body section 104 can include a gel layer of a firm gel, although that need not always be the case. The body section 104 is depicted in FIG. 1 as covering only a portion of hand 108 and not the entirety of hand 108. More specifically, the body section 104 does not cover all digits of the hand 108. Rather, body section 104 covers a portion of the dorsum 116 of the hand (e.g., back of the hand), such as a portion including a region defined by the dorsum of the thumb 110, the dorsum of the index finger 112, and the wrist 122.

As depicted, the body section 104 includes a thumb extension 126 that extends down a length of the thumb 110 but ends prior to the distal end of the thumb 110. In some cases, however, the body section 104 can extend over the distal end of the thumb 110. As depicted, body section 104 includes an index finger extension 128 that extends down a length of the index finger 112 but ends prior to the distal end of the index finger 112. In some cases, however, the body section 104 can extend over the distal end of the index finger. In some cases, the thumb extension 126 can extend to the metacarpophalangeal joint or the interphalangeal joint. In some cases, the index finger extension 128 can extend to the metacarpophalangeal joint, the proximal interphalangeal joint, or the distal interphalangeal joint. It has been found to be especially useful for the thumb extension 128 to extend to at or near the interphalangeal joint while the index finger extension 128 extends past the distal interphalangeal joint.

The medical hand guard 100 can include a dorsal absorption layer 102. The dorsal absorption layer 102 can be coupled to and/or incorporated into the body section 104. In some cases, however, the dorsal absorption layer 102 can be used without a body section 104. The dorsal absorption layer 102 can be shaped to conform to a dorsal thenar space 114 of the hand 108. The dorsal absorption layer 102 can cover a portion of the dorsum 116 of the hand that includes the dorsal thenar space 114, such as a portion including a region defined by the dorsum of the thumb 110, the dorsum of the index finger 112, and the wrist 122. Generally, the dorsal absorption layer 102 extends in a proximal direction only as far as the carpal bones of the hand 108, although that need not always be the case. The dorsal absorption layer 102 can include a thumb extension 130 that extends along a side and/or dorsum of the thumb 110 and an index finger extension 132 that extends along a side and/or dorsum of the index finger 112. The dorsal absorption layer 102 can be shaped to not extend over or into the webspace between the thumb 110 and index finger 112. In other words, the perimeter of the dorsal absorption layer 102 can be spaced apart from the edge of the webspace between the thumb 110 and index finger 112, sometimes colloquially known as the purlicue.

The thumb extension 130 of the absorption layer 102 can extend down a length of the thumb 110 to a location at or prior to the end of thumb extension 126 of the body section 104. The index finger extension 132 of the absorption layer 102 can extend down a length of the index finger 112 to a location at or prior to the end of the index finger extension 128 of the body section 104. In some cases, the thumb extension 130 can extend to the metacarpophalangeal joint or the interphalangeal joint. In some cases, the index finger extension 132 can extend to the metacarpophalangeal joint, the proximal interphalangeal joint, or the distal interphalangeal joint. It has been found to be especially useful for the thumb extension 130 to extend to at or near the metacarpophalangeal joint while the index finger extension 128 extends to a point between the metacarpophalangeal joint and the proximal interphalangeal joint.

As described in further detail herein, the absorption layer 102 can be made of a gel. The gel can include a multi-part gel that includes a semi-fluid gel surrounded by a firm gel. The term “firm gel” as used herein is inclusive of a gel that has a higher viscosity and/or yield stress than the semi-fluid gel. The term “firm gel” can be inclusive of a gel that has a sufficient viscosity to resist flow. The term “semi-fluid gel” can be inclusive of a gel that has a lower viscosity and/or yield stress than the firm gel. The term “semi-fluid gel” can be inclusive of a gel that has a sufficiently low viscosity to permit flow or plastic deformation.

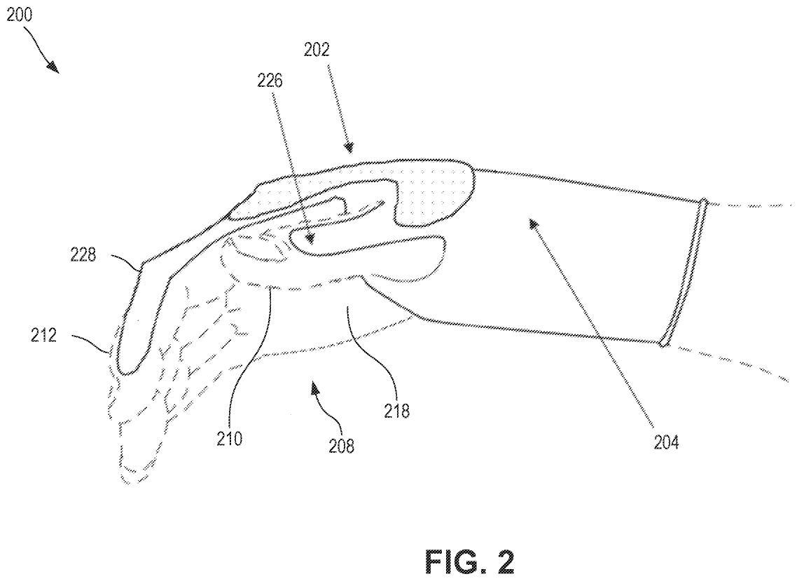

FIG. 2 is a side view of a medical hand guard 200 with a dorsal absorption layer 202, according to certain aspects of the present disclosure. The medical hand guard 200 can be medical hand guard 100 of FIG. 1.

The body section 204 is seen covering a portion of hand 208. The thumb extension 226 of the body section 204 is seen extending along a length of thumb 210 along a dorsal portion of the thumb 210. The index extension 228 of the body section 204 is seen extending along a length of index finger 212 along a dorsal portion and a side portion of the index finger 212.

The dorsal absorption layer 202 is shown coupled to the body section 204. The body section 204 can be coupled to the skin of hand 208, such as via an adhesive layer. Because of the shape of the body section 204 depicted in FIG. 2, the palm 218 of hand 208 remains open and uncovered by the body section 204.

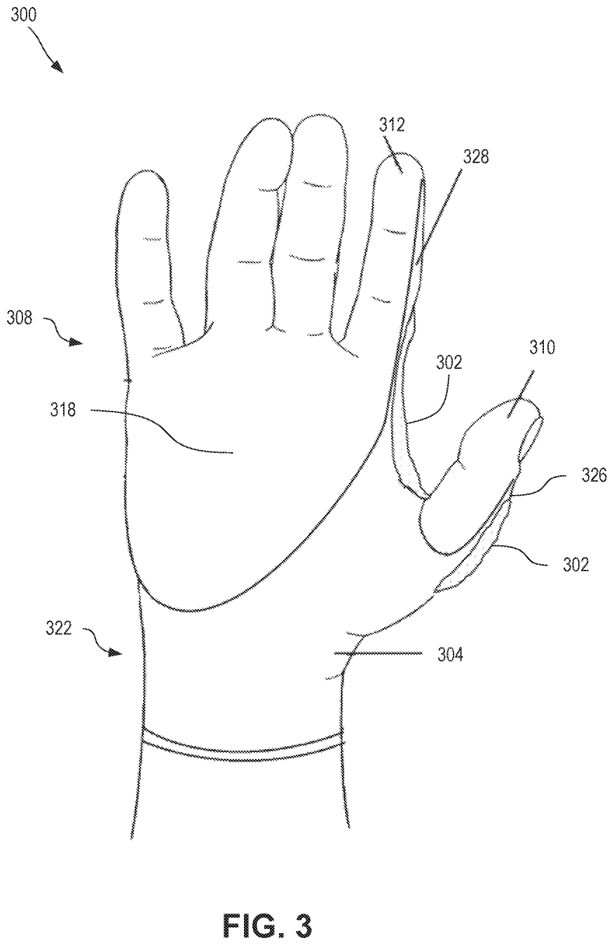

FIG. 3 is a bottom view of a medical hand guard 300 with a dorsal absorption layer 302, according to certain aspects of the present disclosure. The medical hand guard 300 can be medical hand guard 200 of FIG. 2.

The body section 304 is seen covering a portion of hand 308. The thumb extension 326 of the body section 304 is seen extending along a length of thumb 310 along a dorsal portion of the thumb 310. The index extension 328 of the body section 304 is seen extending along a length of index finger 312 along at least side portion of the index finger 312.

The dorsal absorption layer 302 is shown coupled to the body section 304. The body section 304 can be coupled to the skin of hand 308, such as via an adhesive layer. Because of the shape of the body section 304 depicted in FIG. 3, the palm 318 of hand 308 remains open and uncovered by the body section 304.

In some cases, the body section 304 can include a portion wrapping around a wrist 322 of the user, providing a further option for additionally coupling the body section 304 to the user.

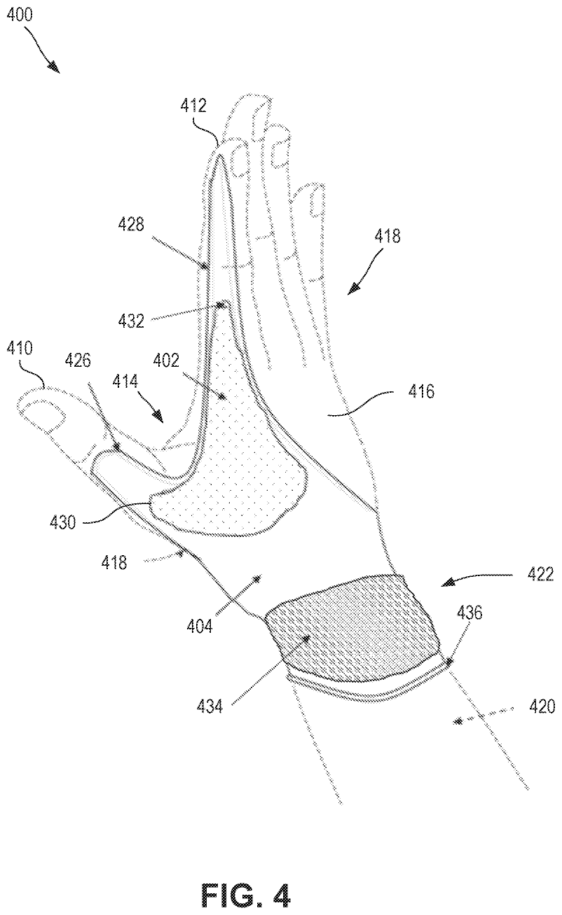

FIG. 4 is an axonometric projection of a medical hand guard 400 with a dorsal absorption layer 402 and a wrist layer 434, according to certain aspects of the present disclosure. The medical hand guard 400 can be similar to medical hand guard 100 of FIG. 1, except with the presence of a wrist layer 434.

The medical hand guard 400 is designed to be worn on a hand 408 of a user. The hand 408 can include a thumb 410 and index finger 412. The hand 408 can be coupled to an arm 420 by a wrist 422. The medical hand guard 400 is depicted as adhered to the hand 408 using an adhesive layer (not shown). In other cases, however, the medical hand guard 400 can be adhered to a standard medical glove or incorporated into a medical glove.

The medical hand guard 400 can include a body section 404. The body section 404 can be made of a elastomeric material, such as a material similar to that of a standard medical glove (e.g., latex or nitrile). The body section 404 can have a thickness at or around 4 to 2 mils, although other thicknesses can be used. In some cases, the body section 404 can include a gel layer of a firm gel, although that need not always be the case. The body section 404 is depicted in FIG. 4 as covering only a portion of hand 408 and not the entirety of hand 408. More specifically, the body section 404 does not cover all digits of the hand 408. Rather, body section 404 covers a portion of the dorsum 416 of the hand (e.g., back of the hand), such as a portion including a region defined by the dorsum of the thumb 410, the dorsum of the index finger 412, and the wrist 422.

As depicted, the body section 404 includes a thumb extension 426 that extends down a length of the thumb 410 but ends prior to the distal end of the thumb 410. In some cases, however, the body section 404 can extend over the distal end of the thumb 410. As depicted, body section 404 includes an index finger extension 428 that extends down a length of the index finger 412 but ends prior to the distal end of the index finger 412. In some cases, however, the body section 404 can extend over the distal end of the index finger. In some cases, the thumb extension 426 can extend to the metacarpophalangeal joint or the interphalangeal joint. In some cases, the index finger extension 428 can extend to the metacarpophalangeal joint, the proximal interphalangeal joint, or the distal interphalangeal joint. It has been found to be especially useful for the thumb extension 428 to extend to at or near the interphalangeal joint while the index finger extension 428 extends past the distal interphalangeal joint.

The medical hand guard 400 can include a dorsal absorption layer 402. The dorsal absorption layer 402 can be coupled to and/or incorporated into the body section 404. In some cases, however, the dorsal absorption layer 402 can be used without a body section 404. The dorsal absorption layer 402 can be shaped to conform to a dorsal thenar space 414 of the hand 408. The dorsal absorption layer 402 can cover a portion of the dorsum 416 of the hand that includes the dorsal thenar space 414, such as a portion including a region defined by the dorsum of the thumb 410, the dorsum of the index finger 412, and the wrist 422. Generally, the dorsal absorption layer 402 extends in a proximal direction only as far as the carpal bones of the hand 408, although that need not always be the case. The dorsal absorption layer 402 can include a thumb extension 430 that extends along a side and/or dorsum of the thumb 410 and an index finger extension 432 that extends along a side and/or dorsum of the index finger 412. The dorsal absorption layer 402 can be shaped to not extend over or into the webspace between the thumb 410 and index finger 412. In other words, the perimeter of the dorsal absorption layer 402 can be spaced apart from the edge of the webspace between the thumb 410 and index finger 412, sometimes colloquially known as the purlicue.

The thumb extension 430 of the absorption layer 402 can extend down a length of the thumb 410 to a location at or prior to the end of thumb extension 426 of the body section 404. The index finger extension 432 of the absorption layer 402 can extend down a length of the index finger 412 to a location at or prior to the end of the index finger extension 428 of the body section 404. In some cases, the thumb extension 430 can extend to the metacarpophalangeal joint or the interphalangeal joint. In some cases, the index finger extension 432 can extend to the metacarpophalangeal joint, the proximal interphalangeal joint, or the distal interphalangeal joint. It has been found to be especially useful for the thumb extension 430 to extend to at or near the metacarpophalangeal joint while the index finger extension 428 extends to a point between the metacarpophalangeal joint and the proximal interphalangeal joint.

As described in further detail herein, the absorption layer 402 can be made of a gel. The gel can include a multi-part gel that includes a semi-fluid gel surrounded by a firm gel.

The wrist layer 434 can cover at least a portion of wrist 422. In some cases, the wrist layer 434 can entirely surround wrist 422 (e.g., surround a circumference of wrist 422). The wrist layer 434 can be coupled to and/or incorporated into the body section 404, similarly to the dorsal absorption layer 402. The wrist layer 434 can be made of or include a gel, such as a firm gel. The gel of the wrist layer 434 can separated into segments, such as parallel segments, to facilitate flexion of the wrist 422 while still providing pressure to the wrist 422 to reduce pain and improve comfort.

In some cases, the body section 404 can include a cuff 436 to help secure the body section 404 to arm 420. The cuff 436 can be similar to a cuff on a standard medical glove. The elastomeric material used for body section 404 can permit the user to move the wrist layer 434 distally or proximally along the wrist 422 and/or arm 420, permitting the wrist layer 434 to be positioned at a desired location for maximum dexterity and/or comfort. In some cases, when no body section 404 is used, or when the wrist layer 434 is otherwise coupled directly to the skin of a user or directly to a medical glove, the wrist layer 434 can be placed on the skin of the user or on the medical glove in a desired location to achieve desired dexterity and/or comfort.

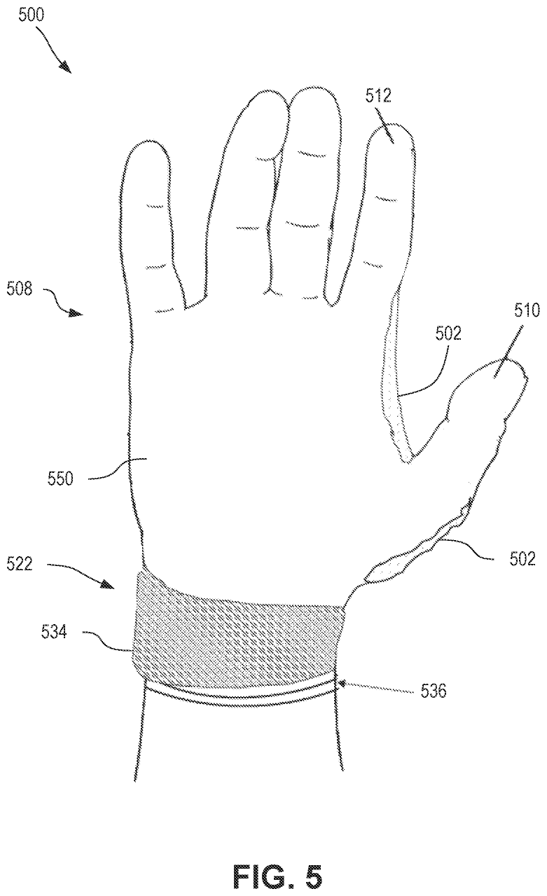

FIG. 5 is a bottom view of a medical hand guard 500 including a dorsal absorption layer 502 and a wrist layer 534 coupled to a medical glove 550, according to certain aspects of the present disclosure. The medical hand guard 500 of FIG. 5 can be similar to medical hand guard 400 of FIG. 4, although with the dorsal absorption layer 502 and wrist layer 534 being directly coupled to a medical glove 550 rather than being coupled to a body layer.

The hand 508 is depicted as wearing a standard medical glove 550 with a cuff 536. The medical glove 550 covers all of the digits of the hand 508, including the index finger 512 and thumb 510. A dorsal absorption layer 502 has been coupled to the medical glove 550, such as via an adhesive (e.g., a pressure-activated adhesive coupled to a bottom side of the dorsal absorption layer 502 and exposed by removing a protective film). The dorsal absorption layer 502 can be positioned to cover the dorsal thenar space of the hand 508. The dorsal absorption layer 502 can otherwise be the same as or similar to any dorsal absorption layer disclosed herein, such as dorsal absorption layer 102 of FIG. 1.

The wrist layer 534 has been coupled to the medical glove 550, such as via an adhesive (e.g., a pressure-activated adhesive coupled to a bottom side of the wrist layer 534 and exposed by removing a protective film). The wrist layer 534 can be positioned to cover the wrist 522, such as a portion of the wrist 522 or an entirety of the wrist 522. In some cases, the wrist layer 534 can be designed to overlap. The wrist layer 534 can otherwise be the same as or similar to any wrist layer disclosed herein, such as wrist layer 434 of FIG. 4.

While the wrist layer 534 is depicted as being coupled to medical glove 550, in other cases, wrist layer 534 can be shaped like a bracelet and merely slid onto a wrist 522 and worn under medical glove 550.

The dorsal absorption layer 502 and wrist layer 534 are depicted as being coupled to a medical glove 550. In some cases, the dorsal absorption layer 502 and wrist layer 534 can be coupled to medical glove 550 through attachment (e.g., via adhesion) after the medical glove 550 has been donned by the medical personnel. In other cases, the dorsal absorption layer 502 and wrist layer 534 can be coupled to and/or otherwise incorporated in medical glove 550 prior to donning of the medical glove 550 (e.g., immediately prior to donning the medical glove 550 during preparation for a medical procedure or during manufacture of the medical glove).

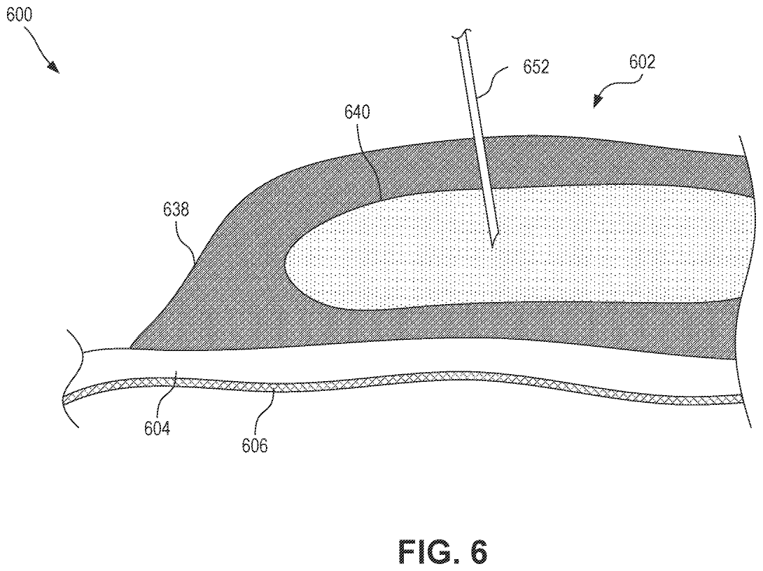

FIG. 6 is a cross-sectional view of a portion of a medical hand guard 600 and dorsal absorption layer 602, according to certain aspects of the present disclosure. The dorsal absorption layer 602 can be any suitable dorsal absorption layer disclosed herein, such as dorsal absorption layer 102 of FIG. 1. For illustrative purposes, a medical sharps object 652 (e.g., a hypodermic needle) is depicted as being inserted into the dorsal absorption layer 602.

The dorsal absorption layer 602 is depicted as being coupled to a body section 604. The body section 604 includes a top surface supporting the dorsal absorption layer 602 and an opposing bottom surface coupled to an adhesive layer 606. The adhesive layer 606 can be used to couple the medical hand guard 600 to the skin of a user or to a medical glove. In some cases, however, the dorsal absorption layer 602 can be directly coupled to an adhesive layer, which can be used to directly couple the dorsal absorption layer 602 to the skin of a user or to a medical glove.

The dorsal absorption layer 602 can include gel. The gel can be a multi-part gel that includes a semi-fluid gel 640 surrounded by a firm gel 638. The multi-part gel can absorb some of the insertion force from the medical sharps object 652, thus providing resistance to insertion of the medical sharps object 652 and giving additional time before the medical sharps object 652 would pass through the dorsal absorption layer 602 to the skin of the user. This additional time can be important to giving sufficient time for the handler of the medical sharps object 652 to withdraw the medical sharps object 652 and/or for the user wearing the dorsal absorption layer 602 to pull away from the medical sharps object 652.

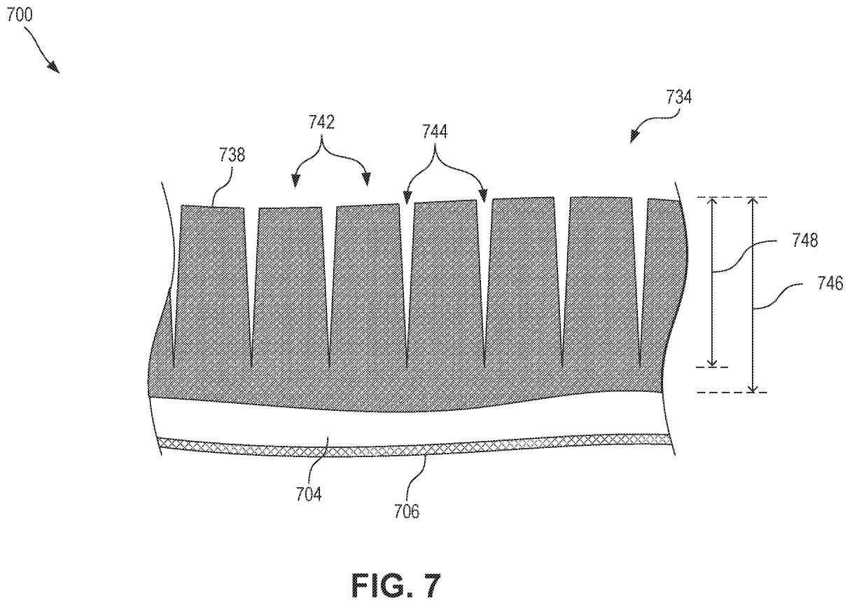

FIG. 7 is a cross-sectional view of a portion of a medical hand guard 700 and wrist layer 754, according to certain aspects of the present disclosure. The wrist layer 754 can be any suitable wrist layer as disclosed herein, such as wrist layer 434 of FIG. 4.

The wrist layer 734 is depicted as being coupled to a body section 704. The body section 704 includes a top surface supporting the dorsal absorption layer 702 and an opposing bottom surface coupled to an adhesive layer 706. The adhesive layer 706 can be used to couple the medical hand guard 700 to the skin of a user or to a medical glove. In some cases, however, the wrist layer 734 can be directly coupled to an adhesive layer, which can be used to directly couple the wrist layer 734 to the skin of a user or to a medical glove.

The wrist layer 734 can include gel 738, such as a firm gel. The gel 738 can include segments 742 separated by separations 744. Separations can extend from a top (e.g., outer) surface of the wrist layer 734 towards a bottom (e.g., inner) surface of the wrist layer 734. As depicted in FIG. 7, the separations 744 extend for a separation depth 748 that is less than the full depth 746 of the wrist layer 734. However, in some cases, the separations 744 can extend through the entire full depth 746 of the wrist layer 734. Separations 744 can be generally parallel with one another. In some cases, separations 744 can be formed by removing a portion of gel 738. In other cases, however, separations 744 can be formed during initial forming of gel 734. Separations 744 can have any suitable cross section, such as generally triangular in shape, as depicted in FIG. 7.

As depicted in FIG. 7, the cross-sectional view is taken along a cross section perpendicular to the direction of separations 744. For example, if a separation 744 is a straight cut in gel 738 extending for a length, the cross-sectional view is taken along a cross section perpendicular to the length of that straight cut. The direction of separations 744 can be shaped to facilitate wrist flexion while still providing the protective and comfort-related benefits. The direction of separations 744 can be angled with respect to an axial line extending through the user's arm. This angle can be 45°, at or less than 45°, or at or less than any of 40°, 35°, 30°, 25°, 20°. 15°, 10°, or 5°. In some cases, the direction of separations 744 can be parallel with respect to an axial line extending through the user's arm.

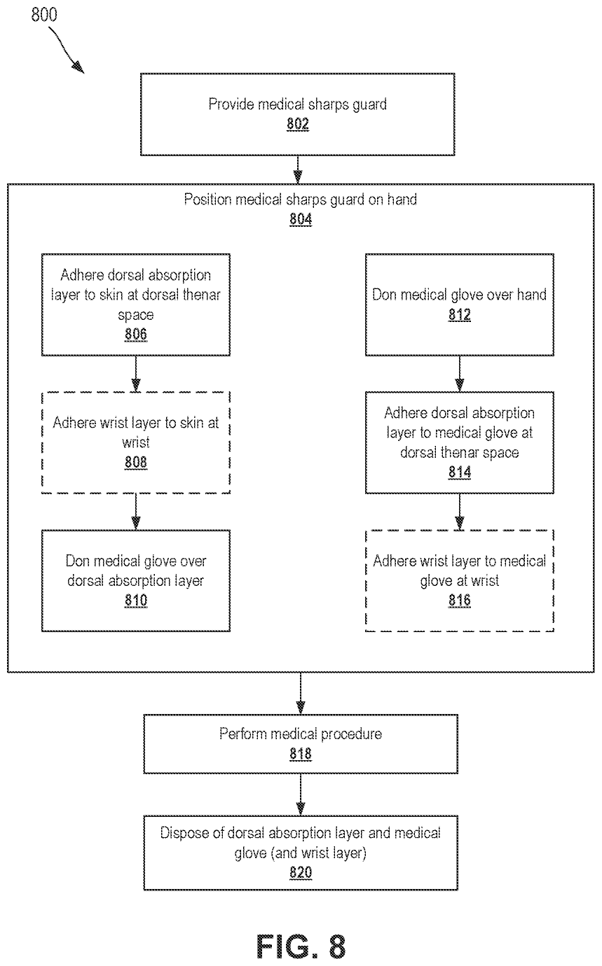

FIG. 8 is a flowchart depicting a process 800 for using a medical hand guard, according to certain aspects of the present disclosure. At block 802, a medical hand guard can be provided. The medical hand guard can include a dorsal absorption layer and optionally a wrist layer. The dorsal absorption layer can be provided on its own (e.g., a dorsal absorption layer directly coupled to an adhesive layer) or coupled to a body section (e.g., a dorsal absorption layer coupled to an adhesive layer through a body section). When the wrist layer is included, the wrist layer can be coupled to the same body section as the dorsal absorption layer, to a separate body section, or separately on its own (e.g., a separate wrist layer directly coupled to an adhesive layer). The medical hand guard can be provided in a sterile condition.

Dorsal absorption layer can be any suitable dorsal absorption layer disclosed herein, such as dorsal absorption layer 102 of FIG. 1. The wrist layer can be any suitable wrist layer disclosed herein, such as wrist layer 434 of FIG. 4.

At block 804, the medical hand guard is positioned on the hand. Positioning the medical hand guard on the hand can occur in different fashions depending on the style of medical hand guard. Positioning the medical hand guard on the hand at block 804 can include positioning the dorsal absorption layer over a dorsal thenar space on the hand. Positioning the medical hand guard on the hand at block 804 can optionally include positioning the wrist layer at and/or around a wrist associated with the hand.

In a first case, positioning the medical hand guard on the hand at block 804 can include donning a medical glove having the medical hand guard incorporated therein.

In a second case, positioning the medical hand guard on the hand at block 804 includes adhering the dorsal absorption layer to the skin of the hand at a dorsal thenar space at block 806, optionally adhering the wrist layer to the skin at the wrist at block 808, and donning a medical glove over the dorsal absorption layer at block 810.

In some cases, adhering a dorsal absorption layer to the skin of the hand at block 806 includes removing a protective film from an adhesive layer and adhering the adhesive layer to the skin of the user. In some cases, the adhesive layer can be coupled directly to the dorsal absorption layer, although in other cases the adhesive layer can be coupled to the dorsal absorption layer via a body section. In some cases, adhering the wrist layer to the skin at block 808 includes using the same adhesive layer from block 806, such as when both the dorsal absorption layer and wrist guard are coupled to a single body section. In other cases, adhering the wrist layer to the skin at block 808 includes using an adhesive layer directly coupled to the wrist layer.

In a third case, positioning the medical hand guard on the hand at block 804 includes donning a medical glove over the hand at block 812, adhering the dorsal absorption layer to the medical glove at block 814, and optionally adhering a wrist layer to the medical glove at the wrist 816.

In some cases, adhering a dorsal absorption layer to the medical glove at block 814 includes removing a protective film from an adhesive layer and adhering the adhesive layer to the medical glove. In some cases, the adhesive layer can be coupled directly to the dorsal absorption layer, although in other cases the adhesive layer can be coupled to the dorsal absorption layer via a body section. In some cases, adhering the wrist layer to the medical glove at block 816 includes using the same adhesive layer from block 814, such as when both the dorsal absorption layer and wrist guard are coupled to a single body section. In other cases, adhering the wrist layer to the medical glove at block 816 includes using an adhesive layer directly coupled to the wrist layer.

At block 818, the medical procedure can be performed. At block 820, the dorsal absorption layer and medical glove, and optional wrist layer if used, can be disposed.

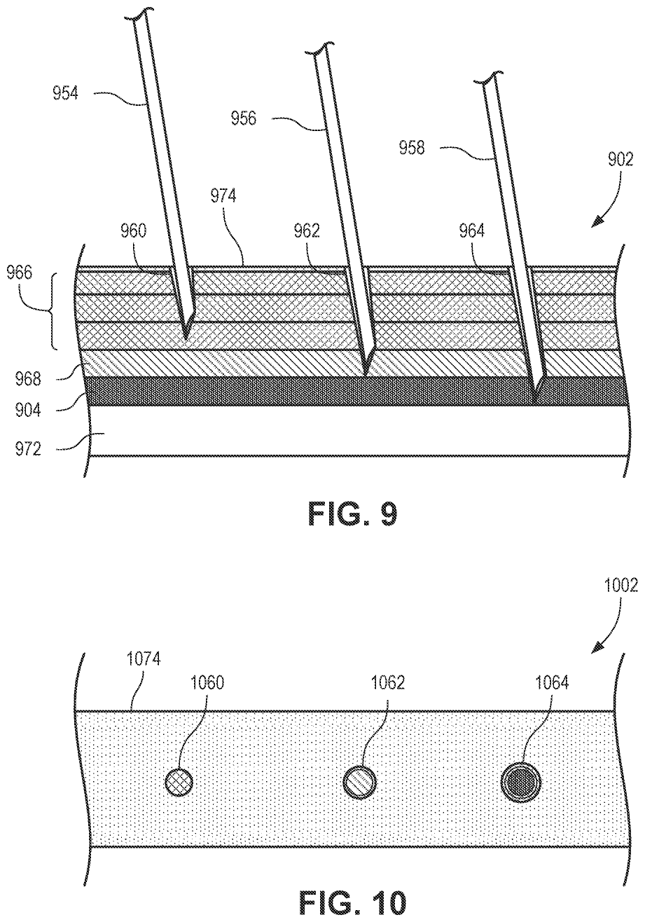

FIG. 9 is a schematic cross-sectional view of a set of a needles 954, 956, 958 puncturing a dorsal absorption layer 902, according to certain aspects of the present disclosure. The dorsal absorption layer 902 can be any suitable dorsal absorption layer, such as dorsal absorption layer 102 of FIG. 1.

The dorsal absorption layer 902 includes a top surface sublayer 974, three outer sublayers 966, and one inner sublayer 968, although any number of layers can be used. The dorsal absorption layer 902 is depicted as being disposed on top of a body section 904 of a medical hand guard (e.g., a sterile glove), which is disposed on top of the user's skin 972. The dorsal absorption layer 902 can be manufactured to be integral with the body section 904 or can be manufactured as a separate element that can be adhered to the body section 904.

In some cases, the top surface sublayer 974, the outer sublayers 966, and/or the inner sublayer 968 can be made of the same material of which the body section 904 is made, or a similar material. For example, if the body section 904 is made of nitrile, one or more of the sublayers 974, 966, 968 may be made out of nitrile or a similar material. The presence of multiple sublayers 974, 966, 968 can cause the region of the medical hand guard occupied by the dorsal absorption layer 902 to be thicker than other regions of the body section 904. This thickening can help resist ingress of sharps, such as needles 954, 956, 958.

In some cases, one or more of the sublayers 974, 966, 968 can be made of gel, such as described in further detail herein (e.g., semi-fluid gel 640 of FIG. 6).

In some cases, one or more of the sublayers 974, 966, 968 of the dorsal absorption layer 902, and optionally the body section 904, can have different visual characteristics, such as differing colors, textures, and the like. For example, the dorsal absorption layer 902 depicted in FIG. 9 can include a top sublayer 974 that is a first color (e.g., blue), a set of outer sublayers 966 that are a second color (e.g., purple), and an inner sublayer 968 that is a third color (e.g., red). In some cases the body section 904 can be a fourth color (e.g., black) or the same color as the top sublayer 974 (e.g., blue). Any other color combinations can be used, and in some cases other visually distinguishing features can be used other than or in addition to color (e.g., texture, reflectivity, and the like). In some cases, each sublayer can be opaque, although that need not always be the case. In some cases, the colors of layers can be selected such that it is easy to distinguish when a layer has been breached.

Each of the sublayers 974, 966, 968 can be arranged such that sublayers closer to the user's skin 972 are visually occluded by the immediately adjacent sublayer further from the user's skin 972. In some cases, each sublayer can be opaque, although that need not always be the case. In some cases, the colors or other visual characteristics of subsequent layers can be selected such that it is easy to distinguish when a layer has been breached. For example, if the inner sublayer 968 has a pattern that consists of parallel lines, the outer sublayers 966 may have a pattern that consists of parallel lines that are perpendicular to those of the inner sublayer 968. Thus, even if the inner sublayer 968 and outer sublayers 966 are non-opaque (e.g., translucent), the presence of crossing lines is indicative that the outer sublayers 966 have not been fully breached, however if only parallel lines are visible in an opening, it can be assumed that the outer sublayers 966 have been breached and the inner sublayer 968 is now exposed.

The different visual characteristics of different sublayers 974,966,968 allow a user to quickly and easily see how deep an opening exists in the dorsal absorption layer 902, such as after a puncture by a sharp. Using the example colors from above, if a user accidentally punctures the dorsal absorption layer 902 and sees the color of the material in the hole is purple and no red is seen, it can be quickly assumed that the puncture passed through the top sublayer 974 and into the outer sublayers 966, but not up to the inner sublayer 968.

As depicted in FIG. 9, needle 954 has entered the dorsal absorption layer 902 and created opening 960. Opening 960 passes through the top surface sublayer 974 and into the outer sublayers 966, without passing through the outer sublayers 966. Thus, when the needle 954 is removed, the user looking into the opening 960 will see the outer sublayers 966, but will not see the inner sublayer 968 or the body section 904. If the outer sublayer 966 has a particular color (e.g., purple), that color will be visible through the opening 960, whereas the color of any deeper layers, which were not punctured, would not be visible.

As depicted in FIG. 9, needle 956 has entered the dorsal absorption layer 902 and created opening 962. Opening 962 passes through the top surface sublayer 974 and the outer sublayers 966, and into but not through the inner sublayer 968. Thus, when the needle 956 is removed, the user looking into the opening 962 will see the inner sublayer 968, and potentially the outer sublayers 966, but will not see the body section 904. If the inner sublayer 968 has a particular color (e.g., red), that color will be visible through the opening 962, whereas the color of any deeper layers (e.g., the body section 904), which were not punctured, would not be visible.

As depicted in FIG. 9, needle 958 has entered the dorsal absorption layer 902 and created opening 964. Opening 964 passes through the top surface sublayer 974, the outer sublayers 966, and the inner sublayer 968, stopping within the body section 904. Thus, when the needle 958 is removed, the user looking into the opening 964 will see body section 904, and potentially the inner sublayer 968 and the outer sublayers 966, but will not see the user's skin 972. If the body section 904 has a particular color (e.g., black or blue), that color will be visible through the opening 964, whereas the color of the user's skin would not be visible.

While described with reference to a dorsal absorption layer 902, similar multilayer structures as disclosed with reference to FIG. 9 can also apply to a wrist layer (e.g., wrist layer 434 of FIG. 4).

FIG. 10 is a schematic top view of a dorsal absorption layer 1002 after being punctured by a set of needles, according to certain aspects of the present disclosure. The dorsal absorption layer 1002 can be dorsal absorption layer 902 of FIG. 9 after removal of the needles (e.g., needles 954, 956, 958 of FIG. 9).

The top surface sublayer 1074 is seen in the top view. For areas without an opening (e.g., due to sharps puncture), the dorsal absorption layer 902 will appear as the color and texture of the top surface sublayer 1074. However, when a puncture or other incident creates an opening (e.g., openings 1060, 1062, 1064), one or more of the layers below the top surface sublayer 1074 may be seen. When those layers have different, distinguishable visual characteristics or features, it can be readily apparent how deep the opening extends into the dorsal absorption layer 902.

Opening 1060 is similar to opening 960 of FIG. 9. The opening 1060 is a result of an object (e.g., needle 954 of FIG. 9) entering the dorsal absorption layer 1002 only as deep as into, but not through, the outer sublayers. Thus, the visual characteristics of the outer sublayers are visible through opening 1060.

Opening 1062 is similar to opening 962 of FIG. 9. The opening 1062 is a result of an object (e.g., needle 956 of FIG. 9) entering the dorsal absorption layer 1002 only as deep as into, but not through, the inner sublayers. Thus, the visual characteristics of the inner sublayer are visible through opening 1062. In some cases, the visual characteristics of the outer sublayers may also be partially visible.

Opening 1064 is similar to opening 964 of FIG. 9. The opening 1064 is a result of an object (e.g., needle 958 of FIG. 9) entering the dorsal absorption layer 1002 only as deep as into, but not through, the body section (e.g., body section 904 of FIG. 9). Thus, the visual characteristics of the body section are visible through opening 1064. In some cases, the visual characteristics of the inner sublayer and/or outer sublayers may also be partially visible.

While described with reference to a dorsal absorption layer 1002, similar multilayer structures as disclosed with reference to FIG. 10 can also apply to a wrist layer (e.g., wrist layer 434 of FIG. 4).

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the invention. Thus, the breadth and scope of the present invention should not be limited by any of the above described embodiments.

Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including.” “includes,” “having.” “has.” “with,” or variants thereof, are used in either the detailed description and/or the claims, such terms are intended to be inclusive in a manner similar to the term “comprising.”

As used below, any reference to a series of examples is to be understood as a reference to each of those examples disjunctively (e.g., “Examples 1-4” is to be understood as “Examples 1, 2, 3, or 4”).

Example 1 is a medical hand guard, comprising: a body section shaped to cover at least a portion of an index finger of a hand of a user and at least a portion of a wrist of the user, the body section having a top surface and a bottom surface; a dorsal absorption layer coupled to the top surface of the body section, wherein the dorsal absorption layer comprises gel, and wherein the dorsal absorption layer covers at least a dorsal thenar space of the hand adjacent a thumb and the index finger of the hand; and an adhesive layer coupled to the bottom surface of the body section.

Example 2 is the medical hand guard of example(s) 1, wherein the gel of the dorsal absorption layer includes a semi-fluid gel material surrounded by a firm gel material.

Example 3 is the medical hand guard of example(s) 1 or 2, wherein the gel of the dorsal absorption layer has a thickness between 2.5 mils and 4.5 mils.

Example 4 is the medical hand guard of example(s) 1-3, wherein the dorsal absorption layer further covers at least a portion of a dorsal region of the index finger and at least a portion of a dorsal region of the thumb.

Example 5 is the medical hand guard of example(s) 1-4, further comprising a wrist layer, the wrist layer comprising additional gel, wherein the wrist layer covers at least a portion of the wrist of the user.

Example 6 is the medical hand guard of example(s) 5, wherein the additional gel of the wrist layer includes an additional firm gel material.

Example 7 is the medical hand guard of example(s) 5 or 6, wherein the wrist layer covers a circumference of the wrist of the user.

Example 8 is the medical hand guard of example(s) 5-7, wherein the additional gel comprises a set of parallel slices to facilitate wrist flexion.

Example 9 is the medical hand guard of example(s) 1-8, wherein the body section comprises an elastomeric material having a thickness between 0.5 mil to 2.5 mil.

Example 10 is the medical hand guard of example(s) 1-9, further comprising a surgical glove covering the body section and dorsal absorption layer, wherein the surgical glove compresses the gel of the dorsal absorption layer against the body section.

Example 11 is the medical hand guard of example(s) 1-10, further comprising a surgical glove for covering all digits of the hand, wherein the surgical glove is coupled to the body section via the adhesive layer.

Example 12 is the medical hand guard of example(s) 1-11, wherein the medical hand guard is sterile and disposable.

Example 13 is the medical hand guard of example(s) 1-12, wherein the dorsal absorption layer comprises a plurality of sublayers, wherein the gel defines at least one of the plurality of sublayers, and wherein a first sublayer of the plurality of sublayers has a first visual characteristic that is distinct from a second visual characteristic of a second sublayer of the plurality of sublayers.

Example 14 is the medical hand guard of example 13, wherein the first visual characteristic is a first color and the second visual characteristic is a second color that is visually distinguishable from the first color.

Example 15 is a medical hand guard, comprising: an absorption layer having a top surface and a bottom surface, wherein the absorption layer comprises gel, and wherein the absorption layer is shaped to cover at least a dorsal thenar space of a hand adjacent a thumb and an index finger of the hand; and an adhesive layer coupled to the bottom surface of the absorption layer.

Example 16 is the medical hand guard of example(s) 15, wherein the gel of the absorption layer includes a semi-fluid gel material surrounded by a firm gel material.

Example 17 is the medical hand guard of example(s) 15 or 16, wherein the gel of the absorption layer has a thickness between 2.5 mils and 4.5 mils.

Example 18 is the medical hand guard of example(s) 15-17, wherein the absorption layer is further shaped to cover at least a portion of a dorsal region of the index finger and at least a portion of a dorsal region of the thumb.

Example 19 is the medical hand guard of example(s) 15-18, further comprising a surgical glove for covering all digits of the hand, wherein the surgical glove is coupled to the absorption layer via the adhesive layer.

Example 20 is the medical hand guard of example(s) 15-19, wherein the medical hand guard is sterile and disposable.

Example 21 is the medical hand guard of example(s) 15-20, further comprising a wrist layer, the wrist layer comprising additional gel, wherein the wrist layer covers at least a portion of the wrist of the user.

Example 22 is the medical hand guard of example(s) 21, wherein the additional gel of the wrist layer includes an additional firm gel material.

Example 23 is the medical hand guard of example(s) 21 or 22, wherein the wrist layer covers a circumference of the wrist of the user.

Example 24 is the medical hand guard of example(s) 21-23, wherein the additional gel comprises a set of parallel slices to facilitate wrist flexion.

Example 25 is the medical hand guard of example(s) 15-24, further comprising a surgical glove covering the absorption layer, wherein the surgical glove compresses the gel of the dorsal absorption layer against the hand when worn.

Example 26 is the medical hand guard of example(s) 15-25, wherein the dorsal absorption layer comprises a plurality of sublayers, wherein the gel defines at least one of the plurality of sublayers, and wherein a first sublayer of the plurality of sublayers has a first visual characteristic that is distinct from a second visual characteristic of a second sublayer of the plurality of sublayers.

Example 27 is the medical hand guard of example(s) 26, wherein the first visual characteristic is a first color and the second visual characteristic is a second color that is visually distinguishable from the first color.

Example 28 is a medical hand guard, comprising: a body section shaped to cover at least a portion of an index finger of a hand of a user and at least a portion of a wrist of the user, the body section having a top surface and a bottom surface; a dorsal absorption layer coupled to the top surface of the body section, wherein the dorsal absorption layer comprises a plurality of sublayers, wherein a first sublayer of the plurality of sublayers has a first visual characteristic that is distinct from a second visual characteristic of a second sublayer of the plurality of sublayers, and wherein the dorsal absorption layer covers at least a dorsal thenar space of the hand adjacent a thumb and the index finger of the hand.

Example 29 is the medical hand guard of example(s) 28, wherein the first visual characteristic is a first color and the second visual characteristic is a second color that is visually distinguishable from the first color.

Example 30 is the medical hand guard of example(s) 28 or 29, further comprising an adhesive layer coupled to the bottom surface of the body section.

Example 31 is the medical hand guard of example(s) 28-30, wherein the plurality of sublayers includes at least one gel sublayer.

Example 32 is the medical hand guard of example(s) 31, wherein the at least one gel sublayer includes a semi-fluid gel material surrounded by a firm gel material.

Example 33 is the medical hand guard of example(s) 28-32, wherein the dorsal absorption layer further covers at least a portion of a dorsal region of the index finger and at least a portion of a dorsal region of the thumb.

Example 34 is the medical hand guard of example(s) 28-33, further comprising a wrist layer, the wrist layer comprising a plurality of wrist sublayers, wherein a first wrist sublayer of the plurality of wrist sublayers has a first wrist sublayer visual characteristic that is distinct from a second wrist sublayer visual characteristic of a second wrist sublayer of the plurality of wrist sublayers.

Example 35 is the medical hand guard of example(s) 34, wherein the plurality of wrist sublayers include a set of parallel slices to facilitate wrist flexion.

1: A medical hand guard, comprising:
a body section shaped to cover at least a portion of an index finger of a hand of a user and at least a portion of a wrist of the user, the body section having a top surface and a bottom surface;
a dorsal absorption layer coupled to the top surface of the body section, wherein the dorsal absorption layer comprises gel, and wherein the dorsal absorption layer covers at least a dorsal thenar space of the hand adjacent a thumb and the index finger of the hand; and an adhesive layer coupled to the bottom surface of the body section.

2: The medical hand guard of claim 1, wherein the gel of the dorsal absorption layer includes a semi-fluid gel material surrounded by a firm gel material.

3: The medical hand guard of claim 1, wherein the gel of the dorsal absorption layer has a thickness between 2.5 mils and 4.5 mils.

4: The medical hand guard of claim 1, wherein the dorsal absorption layer further covers at least a portion of a dorsal region of the index finger and at least a portion of a dorsal region of the thumb.

5: The medical hand guard of claim 1, further comprising a wrist layer, the wrist layer comprising additional gel, wherein the wrist layer covers at least a portion of the wrist of the user.

6: The medical hand guard of claim 5, wherein the additional gel of the wrist layer includes an additional firm gel material.

7: The medical hand guard of claim 5, wherein the wrist layer covers a circumference of the wrist of the user.

8: (canceled)

9: The medical hand guard of claim 1, wherein the body section comprises an elastomeric material having a thickness between 0.5 mil to 2.5 mil.

10: (canceled)

11: The medical hand guard of claim 1, further comprising a surgical glove for covering all digits of the hand, wherein the surgical glove is coupled to the body section via the adhesive layer.

12: The medical hand guard of claim 1, wherein the medical hand guard is sterile and disposable.

13: The medical hand guard of claim 1, wherein the dorsal absorption layer comprises a plurality of sublayers, wherein the gel defines at least one of the plurality of sublayers, and wherein a first sublayer of the plurality of sublayers has a first visual characteristic that is distinct from a second visual characteristic of a second sublayer of the plurality of sublayers.

14: The medical hand guard of claim 13, wherein the first visual characteristic is a first color and the second visual characteristic is a second color that is visually distinguishable from the first color.

15: A medical hand guard, comprising:
an absorption layer having a top surface and a bottom surface, wherein the absorption layer comprises gel, and wherein the absorption layer is shaped to cover at least a dorsal thenar space of a hand adjacent a thumb and an index finger of the hand; and an adhesive layer coupled to the bottom surface of the absorption layer.

16: (canceled)

17: The medical hand guard of claim 15, wherein the gel of the absorption layer has a thickness between 2.5 mils and 4.5 mils.

18: The medical hand guard of claim 15, wherein the absorption layer is further shaped to cover at least a portion of a dorsal region of the index finger and at least a portion of a dorsal region of the thumb.

19: The medical hand guard of claim 15, further comprising a surgical glove for covering all digits of the hand, wherein the surgical glove is coupled to the absorption layer via the adhesive layer.

20: The medical hand guard of claim 15, wherein the medical hand guard is sterile and disposable.

21: (canceled)

22: (canceled)

23: (canceled)

24: (canceled)

25: (canceled)

26: (canceled)

27: (canceled)

28: A medical hand guard, comprising:
a body section shaped to cover at least a portion of an index finger of a hand of a user and at least a portion of a wrist of the user, the body section having a top surface and a bottom surface;
a dorsal absorption layer coupled to the top surface of the body section, wherein the dorsal absorption layer comprises a plurality of sublayers, wherein a first sublayer of the plurality of sublayers has a first visual characteristic that is distinct from a second visual characteristic of a second sublayer of the plurality of sublayers, and wherein the dorsal absorption layer covers at least a dorsal thenar space of the hand adjacent a thumb and the index finger of the hand.

29: The medical hand guard of claim 28, wherein the first visual characteristic is a first color and the second visual characteristic is a second color that is visually distinguishable from the first color.

30: (canceled)

31: (canceled)

32: (canceled)

33: The medical hand guard of claim 28, wherein the dorsal absorption layer further covers at least a portion of a dorsal region of the index finger and at least a portion of a dorsal region of the thumb.

34: (canceled)

35: (canceled)
